(19) United States
(12) Patent Application Publication
(10) Pub. No.: US 2012/0070507 A1
(43) Pub. Date: Mar. 22, 2012

(54) Compositions and Methods for the Treatment of Soft Tissue Injury

(75) Inventors: Corinna HOWELL, San Diego, CA (US); Neil HOWELL, San Diego, CA (US)

(73) Assignee: MATRILINEX LLC, San Diego, CA (US)

(21) Appl. No.: 12/884,819

(22) Filed: Sep. 17, 2010

(51) Int. Cl. A61K 35/407; A61K 31/05; A61K 31/618; A61P 9/00; A61P 21/00; A61K 31/137; A61P 29/00; A61K 31/5375; A61K 31/165

(52) U.S. Cl. 424/553; 514/239.2; 514/731; 514/159; 514/627; 514/653

(57) ABSTRACT

Provided herein are compositions and methods for the treatment of soft tissue injury, sports-related injury, physical overuse of muscles, ligaments or joints, and trauma, traumatic stress or sudden jolts on bones and tissues in an individual. Described herein are pharmaceutical compositions comprising a vasoconstrictor and, optionally, a topical analgesic and methods of using said compositions for the treatment of a variety of soft tissue injuries.

### BACKGROUND OF THE INVENTION

Over the counter analgesics and counter-irritants, so-called “sports creams”, are widely used for the relief of muscle pain. This is accomplished by muscle relaxation via topical application of a mixture of topical analgesics (e.g. salicylates, either methyl salicylate or trolamine salicylate) and/or counterirritants (e.g. camphor, menthol) as active ingredients.

For example, the following sports creams contain methyl salicylate as active ingredient—BenGay “Greaseless” (methyl salicylate 15%, menthol 10%), BenGay “Ultra Strength” (methyl salicylate 30%, menthol 10%, camphor 4%), Flexall “Ultra Plus” (methyl salicylate 10%, menthol 16%, camphor 3.1%), Icy Hot “Balm” (methyl salicylate 29%, Menthol 7.6%), Icy Hot “Cream” (methyl salicylate 30%, menthol 10%), Icy Hot “Stick” (methyl salicylate 30%, menthol 10%), Thera-Gesic “Maximum Strength” (methyl salicylate 15%, menthol 1%) and TheraFlex (methyl salicylate; dosage not disclosed).

Sports creams that contain trolamine salicylate include for example, Aspercreme (trolamine salicylate 10%), Myoflex (trolamine salicylate 10%), SportsCreme (trolamine salicylate 10%), ALCiS (trolamine salicylate 10%), FlexPower (trolamine salicylate 10%) and Aspercreme (trolamine salicylate 10%).

Sports creams that contain counterirritants only include, for example, BenGay “Vanishing Scent” (menthol 2.5%), Biofreeze (menthol 3.5%, camphor 0.2%), Flexall 454 (menthol 7%), Flexall Maximum Strength (menthol 16%), Icy Hot Patch (menthol 5%), and Icy Hot Sleeve (menthol 16%).

Many sports creams contain capsaicin as active ingredient including Tiger Balm pain relieving patch (capsaicin, menthol, camphor; dosage not disclosed), Tiger Balm ointment ultra (menthol, camphor, cajuput, clove, cassia, capsaicin and peppermint oil) and Tiger Balm Ultra (menthol, camphor, cajuput, clove, cassia, capsaicin and peppermint oil).

These sports creams do not “heal” the injury, but rather mask the effect of an inflicted injury by minimizing the pain caused by the injury.

### SUMMARY OF THE INVENTION

One embodiment provides a method of treating a soft tissue injury in an individual comprising topical application of a pharmaceutical composition comprising a vasoconstrictor and a topical analgesic.

Another embodiment provides the method wherein the soft tissue injury is a muscular injury, a sports-related injury or a muscle-related low back injury.

Another embodiment provides the method wherein the vasoconstrictor is phenylephrine and the topical analgesic is pramoxine.

Another embodiment provides the method wherein the topically applied pharmaceutical composition further comprises at least one of the following: menthol, methyl salicylate, or capsaicin.

Another embodiment provides the method wherein the topically applied pharmaceutical composition further comprises at least one of the following: aloe barbadebsis leaf extract, benzoic acid, butylated hydroxyanisole, carboxymethylcellulose sodium, cetyl alcohol, citric acid, dexpanthenol, edentate sodium, glyceryl stearate, laureth-23, methylparaben, mineral oil, panthenol, polyoxyl lauryl ether, polyoxyl stearyl ether, propyl gallate, propylene glycol, propylparaben, sodium benzoate, steareth-2, steareth-20, stearyl alcohol, tocopherol, vitamin E, xanthan gum, wintergreen, eucalyptus oil, corn oil, lanolin, mineral oil, thyme oil, or white wax.

One embodiment provides a method of treating muscular injury in an individual comprising topical application of a pharmaceutical composition comprising phenylephrine.

Another embodiment provides the method wherein muscular pain associates with said muscular injury.

One embodiment provides a method for treating sports-related injury in an individual comprising topical application of a pharmaceutical composition comprising phenylephrine.

One embodiment provides a method of treating soft tissue injury in an individual comprising topical application of a pharmaceutical composition comprising phenylephrine.

One embodiment provides a method for treating muscle-related low back injury in an individual comprising topical application of a pharmaceutical composition comprising phenylephrine.

Another embodiment provides the method wherein the topically applied pharmaceutical composition further comprises glycerin.

Another embodiment provides the method wherein the topically applied pharmaceutical composition further comprises white petrolatum.

Another embodiment provides the method wherein the topically applied pharmaceutical composition further comprises pramoxine.

Another embodiment provides the method wherein the topically applied pharmaceutical composition comprises phenylephrine from about 0.05% (w/w) to about 2.0% (w/w).

Another embodiment provides the method wherein the topically applied pharmaceutical composition comprises glycerin from about 10% (w/w) to about 20% (w/w).

Another embodiment provides the method wherein the topically applied pharmaceutical composition comprises white petrolatum from about 10% (w/w) to about 20% (w/w).

Another embodiment provides the method wherein the topically applied pharmaceutical composition comprises pramoxine HCl from about 1.0% (w/w) to about 2.0% (w/w).

Another embodiment provides the method wherein the topically applied pharmaceutical composition further comprises light mineral oil or shark liver oil.

Another embodiment provides the method wherein the topically applied pharmaceutical composition comprises light mineral oil from about 10% (w/w) to about 20% (w/w).

Another embodiment provides the method wherein the topically applied pharmaceutical composition comprises shark liver oil from about 0.5% (w/w) to about 10% (w/w).

Another embodiment provides the method wherein the injury is a muscle injury selected from a muscle tear, muscle pull, muscle bruising, muscle strain or muscle swelling.

Another embodiment provides the method wherein the injury occurs in a muscle selected from:

a. neck muscle comprising omohyoid, sternohyoid, sternocleidomastoid, or trapezius muscle;

b. chest muscle comprising pectoralis major, or pectoralis minor muscle;

c. shoulders muscles comprising deltoid, anterior head, middle head, or posterior head;

d. bicep muscle comprising brachialis, biceps brachil long head, or biceps brachil short head;

e. forearm muscle comprising extensor carpi ulnaris, palmaris longus, extensor pollicis brevis, abductore pollicis longus, extensor pollicis longus, flexor carpi radialis, brachioradialis, or flexor carpi ulnaris;

f. abdominal muscle comprising serratur anterior, external oblique, rectur abdominis, tendinous inscriptions, or external oblique;

g. thigh muscle comprising quadriceps, vastus lateralis, vastus rectus femoris, vastus medialis, vastus intermedialis; sartorius, pectineus, adductor longus, gracilis, or tensor fasciae latae;

h. calf muscle comprising gastrocnemius soleus, tibialis anterior, peroneus longus, extensor digitorum longus, peroneus brevis, or flexor hallucis longus;

i. tricep muscle comprising triceps brachi, triceps lateral head, triceps long head, or triceps medial head;

j. glute muscle comprising gluteus medius, or gluteus maximus; and

k. hamstring muscle comprising liotibial band, biceps femoris, adductor magnus, semitendinosus gracilis, or semimembranosus.

Another embodiment provides the method wherein the muscle-related low back injury occurs in at least one muscle selected from:

a. back muscles comprising teres minor, teres major, intraspinatus, rhomboid major, trapezius, erector spinea, latissimus dorsi, or thoracolumbar fascia;

b. lower back muscles comprising erector spinea, thoracolumbar fascia, external oblique, or gluteus medius; and

c. lower back muscles that are involved in low back pain caused by three types of muscles that support the spine, comprised of extensors (back muscles and gluteal muscles), flexors (abdominal muscles and iliopsoas muscles), obliques and rotators (side muscles).

Another embodiment provides the method wherein the injury is selected from:

a. hematomas associated with trauma-induced pooling of blood under the skin and within or around muscle tissue;

b. muscle bruising and muscle tears in skeletal muscles;

c. low back muscles;

d. bone bruises of the knee;

e. bone bruises of the shin;

f. bone bruises of the foot;

g. bone bruises of the ball of the foot;

h. bone bruises of the heel;

i. bone bruises of the toes;

j. bone bruises of the ankle;

k. sore elbow;

l. tennis elbow;

m. bone bruise of the elbow;

n. sore shoulder; and

o. bone bruise of the shoulder.

Another embodiment provides the method wherein the phenylephrine is present as a pharmaceutically acceptable salt.

Another embodiment provides the method wherein the pharmaceutically acceptable salt is phenylephrine hydrochloride.

Provided herein are methods of treating a sports-related injury in an individual comprising topical application of a pharmaceutical composition comprising a vasoconstrictor and a topical analgesic.

Another aspect provides methods of treating a muscular pain in an individual comprising topical application of a pharmaceutical composition comprising phenylephrine.

### DETAILED DESCRIPTION OF THE INVENTION

The majority of over-the-counter “sports creams” mask the effect of an inflicted injury by minimizing the pain caused by the injury. In contrast to this current practice, provided herein are compositions and methods that heal the injury and thereby remove the source of pain.

One embodiment provides a method of treating a soft tissue injury in an individual comprising topical application of a pharmaceutical composition comprising a vasoconstrictor and a topical analgesic. Another embodiment provides the method wherein the soft tissue injury is a muscular injury, a sports-related injury or a muscle-related low back injury. Another embodiment provides a method of treating a soft tissue injury wherein said injury is characterized by muscle tear, muscle pull, muscle bruising, muscle strain, or muscle swelling. Another embodiment provides a method of treating a soft tissue injury wherein said injury is characterized by damage or rupture of blood vessels or capillaries.

One embodiment provides a method of treating a soft tissue injury in an individual comprising topical application of a pharmaceutical composition comprising phenylephrine. Another embodiment provides the method wherein the soft tissue injury is a muscular injury, a sports-related injury or a muscle-related low back injury.

Many sports-related injuries result in bruises, also called contusions, which are a type of hematoma of tissue in which capillaries and sometimes venules are damaged by trauma, allowing blood to seep into the surrounding extracellular space. In some instances, bruises involve capillaries at the level of skin, subcutaneous tissue, muscle, or bone. Nerve endings within the affected tissue detect the increased pressure, which, depending on severity and location, may be perceived as pain or pressure. The damaged endothelium (lining) of the affected capillaries releases endothelin, a hormone that causes narrowing of the blood vessel to minimize bleeding. However, as the endothelium is destroyed following the injury, coagulation is initiated and creates a clot to plug the wound, thereby increasing the pain and pressure perceived by nerve endings.

In accordance with the practice of the present invention, there are provided methods of treating sports-related injury e.g., hematomas by topical application of a vasoconstrictor constricting blood vessels at the injury site. Hematomas that are effectively treated are associated with trauma-induced pooling of blood, for example, under the skin, small muscle tears in all major muscles of the body (e.g., quadriceps, hamstring, shoulder), bone bruises (e.g., bone bruises of the knee, the shin, the foot, the ball of the foot, the heel, the toes, and the ankle), sore elbow (e.g., tennis elbow an bone bruise of the elbow), sore shoulder (e.g., bone bruise of the shoulder or a muscle tear), and the like.

Another embodiment provides a method of treating a sports-related injury in an individual wherein the sports-related injury includes physical overuse of muscles, ligaments or joints, trauma and traumatic stress; or sudden jolts on bones and tissues comprising topical application of a pharmaceutical composition comprising a vasoconstrictor and a topical analgesic.

### Low Back Pain From Muscle Strain

Acute or short term low back pain generally lasts from days to weeks. Most acute back pain, incuding a muscle strain or tear, is the result of trauma to the lower back cased by damage to the muscles and/or ligaments in the low back. Pain from trauma may be caused by a sports injury, heavy lifting, overstretching, sudden movement, or a sudden jolt or other stress on spinal bones and tissues, causing a sprain, strain or spasm in one of the muscles or ligaments in the back. Low back pain may reflect nerve or muscle irritation or bone lesions. Most low back pain follows injury or trauma to the back. Nearly everyone has low back pain on occasion; men and women are equally affected. The spine is supported by large muscles called the paraspinal muscles. These muscles support both the spinal column as well as the weight of the upper body. These muscles, ligaments, and bones all work together to provide control and strength for nearly all activities. The lumbar spine and its muscles are needed for most all movements and activities. The majority of episodes of acute lower back pain are caused by damage to the muscles and/or ligaments (which attach bone to bone) in the low back, caused by some combination of overuse, muscle irritation, muscle strain, muscle ruptures, muscle tears, and injury to muscles and ligaments. Low back pain can be severe and is the cause of many emergency room visits each year. A muscle strain happens when the muscle is over-stretched or torn, resulting in damage to the muscle fibers (a pulled muscle). A lumbar sprain happens when ligaments are stretched too far or torn. Ligaments are very tough, fibrous connecting tissues that connect the muscles to the bones and joints. When the muscles or ligaments in the low back are strained or torn, the area around the muscles can become inflamed. With inflammation the muscles in the back can spasm and cause both severe lower back pain and difficulty moving. The symptoms include sharp pain in the back, chronic pain in the back, swelling and bruising over the area of muscle rupture or tear or strain, and difficulty in moving, bending or straightening.

### Bone Bruise of the Knee

During a bone bruise, bleeding occurs under the tough fibrous covering of the bone called the periosteum. It causes pain in the area of the bone but heals eventually as the blood is reabsorbed just like a bruise of the skin. In some instances, the bruising involves the skin and soft tissues as well. The most painful bruises can sometimes take months to heal. What happens in a bone bruise of the knee is a compressive force that pushes the femur and tibia together. When this happens the outer layer of the bone (most likely the femur), which is fibrous, breaks down. This leads to leaking of fluid. The traditional treatment of a bone bruise consists of ice, rest, and pain relief (ibuprofen). The recovery time ranges from days to weeks to several months.

### Bone Bruise of the Shin

A direct blow to the shin, caused for example by a kick from a football tackle or a hockey stick or a racquet, can cause bone damage as there is not much protection from muscle or fat over the shin bone to absorb a direct blow. Bruising and swelling gradually appear and, if the bone is damaged, the swelling is usually greater and the bone will feel painful to the touch.

### Bone Bruise of the Foot

Bone bruises under the ball of the big toe are caused by bruising of tiny bones called sesamoids. Runners are usually affected when running extensively on the balls of their feet through high mileage, or new hillwork, or speedwork, or on hard pavement. Common remedies include icing the foot repeatedly up to four times per day.

### Bone Bruises of the Heel

Bone bruises of the heel occur most frequently in sports that involve jumping and severe or repetitive landing, such as gymnastics or racquet sports where the heel can be landed on suddenly with force. Such injuries can be difficult to treat and are sometimes stubborn and persist for some weeks. Common treatments call for rest until there is no more heel pain, padding of the heel of shoes with a shock absorbing insole or heel pad, and taping of the heel to provide pain relief.

### Bruised Toes

Bruised toes can feel very painful and present with swelling and “black and blue” discolorations. However, they usually appear straight, and the injury which caused the bruise is minor.

### Tennis Elbow

Many different types of injury are often labeled as tennis elbow. Tennis elbow is a strain or inflammation or both of the wrist muscles where they connect to the elbow joint on the outside. Tennis elbow is usually the result of repetitive use either in racquet sports or throwing which over stresses the surrounding structures of the elbow. The damaged area will feel tender over the outside of the elbow, giving not only elbow pain but lower arm and elbow pain.

### Vasoconstrictors And Analgesics

Vasoconstrictors include, for example, amphetamines, antihistamines, methylphenidate, oxymetazoline, phenylephrine, pseudoephedrine, caffeine, tetrahydrozoline hydrochloride, and the like.

An analgesic (also known as a painkiller) is any member of the group of drugs used to relieve pain. Analgesics that are used topically include, for example, ibuprofen, diclofenac, capsaicin, lidocaine, salicylates (e.g., methyl salicylate or trolamine salicylate), pramoxine, menthol, and the like.

### Methods of Treatment

In some embodiments, there are provided methods of treating a sports-related injury in an individual comprising topical application of a pharmaceutical composition comprising a vasoconstrictor and a topical analgesic. In certain embodiments, the vasoconstrictor is phenylephrine and the topical analgesic is pramoxine. In some embodiments, the topically applied pharmaceutical composition further comprises at least one of the following: menthol, methyl salicylate, or capsaicin. In other embodiments, the topically applied pharmaceutical composition further comprises at least one of the following: aloe barbadebsis leaf extract, benzoic acid, butylated hydroxyanisole, carboxymethylcellulose sodium, cetyl alcohol, citric acid, dexpanthenol, edentate sodium, glyceryl stearate, laureth-23, methylparaben, mineral oil, panthenol, polyoxyl lauryl ether, polyoxyl stearyl ether, propyl gallate, propylene glycol, propylparaben, sodium benzoate, steareth-2, steareth-20, stearyl alcohol, tocopherol, vitamin E, xanthan gum, wintergreen, eucalyptus oil, corn oil, lanolin, mineral oil, thyme oil, or white wax.

Some embodiments provide methods of treating muscular pain in an individual comprising topical application of a pharmaceutical composition comprising phenylephrine. In certain embodiments, the muscular pain is associated with muscular injury. In some embodiments, the topically applied pharmaceutical composition further comprises glycerin. In other embodiments, the topically applied pharmaceutical composition further comprises white petrolatum. In other embodiments, the topically applied pharmaceutical composition further comprises pramoxine. In certain embodiments, the topically applied pharmaceutical composition comprises phenylephrine from about 0.05% (w/w) to about 2.0% (w/w). In certain embodiments, the topically applied pharmaceutical composition comprises glycerin from about 10% (w/w) to about 20% (w/w). In certain embodiments, the topically applied pharmaceutical composition comprises white petrolatum from about 10% (w/w) to about 20% (w/w). In certain embodiments, the topically applied pharmaceutical composition comprises pramoxine HC1 from about 1.0% (w/w) to about 2.0% (w/w). In certain embodiments, the topically applied pharmaceutical composition further comprises light mineral oil or shark liver oil. In certain embodiments, the topically applied pharmaceutical composition comprises light mineral oil from about 10% (w/w) to about 20% (w/w). In certain embodiments, the topically applied pharmaceutical composition comprises shark liver oil from about 0.5% (w/w) to about 10% (w/w). In some embodiments, the muscular injury is selected from a muscle tear, muscle pull, muscle bruising, muscle strain, muscle swelling, and the like. In other embodiments, the phenylephrine is present as a pharmaceutically acceptable salt. In certain embodiments, the pharmaceutically acceptable salt is phenylephrine hydrochloride.

In certain embodiments, the muscle injury occurs in a muscle selected from:

a. neck muscle comprising omohyoid, sternohyoid, sternocleidomastoid, or trapezius muscle;

b. chest muscle comprising pectoralis major, or pectoralis minor muscle;

c. shoulders muscles comprising deltoid, anterior head, middle head, or posterior head;

d. bicep muscle comprising brachialis, biceps brachil long head, biceps brachil short head;

e. forearm muscle comprising extensor carpi ulnaris, palmaris longus, extensor pollicis brevis, abductore pollicis longus, extensor pollicis longus, flexor carpi radialis, brachioradialis, or flexor carpi ulnaris;

f. abdominal muscle comprising serratur anterior, external oblique, rectur abdominis, tendinous inscriptions, or external oblique;

g. thigh muscle comprising quadriceps, vastus lateralis, vastus rectus femoris, vastus medialis, vastus intermedialis; sartorius, pectineus, adductor longus, gracilis, or tensor fasciae latae;

h. calf muscle comprising gastrocnemius soleus, tibialis anterior, peroneus longus, extensor digitorum longus, peroneus brevis, or flexor hallucis longus;

i. tricep muscle comprising triceps brachi, triceps lateral head, triceps long head, or triceps medial head;

j. glute muscle comprising gluteus medius, or gluteus maximus; or

k. hamstring muscle comprising liotibial band, biceps femoris, adductor magnus, semitendinosus gracilis, or semimembranosus.

In certain embodiments the muscle-related low back injury occurs in at least one muscle selected from:
a. back muscles comprising teres minor, teres major, intraspinatus, rhomboid major, trapezius, erector spinea, latissimus dorsi, or thoracolumbar fascia;
b. lower back muscles comprising erector spinea, thoracolumbar fascia, external oblique, or gluteus medius; and
c. lower back muscles that are involved in low back pain caused by three types of muscles that support the spine, comprised of extensors (back muscles and gluteal muscles), flexors (abdominal muscles and iliopsoas muscles), obliques and rotators (side muscles).

### Muscle Injuries

In certain embodiments, the injury is selected from:

a. hematomas associated with trauma-induced pooling of blood under the skin and within muscle tissue;

b. muscle bruising and muscle tears in skeletal muscles;

c. lower back pain caused by three types of muscles that support the spine: extensors (back muscles and gluteal muscles), flexors (abdominal muscles and iliopsoas muscles), obliques and rotators (side muscles);

d. bone bruises of the knee;

e. bone bruises of the shin;

f. bone bruises of the foot;

g. bone bruises of the ball of the foot;

h. bone bruises of the heel;

i. bone bruises of the toes;

j. bone bruises of the ankle;

k. sore elbow;

l. tennis elbow;

m. bone bruise of the elbow;

n. sore shoulder; or

o. bone bruise of the shoulder.

In some embodiments provided herein are methods for treating a sports-related injury in an individual comprising topical application of a pharmaceutical composition comprising phenylephrine. In other embodiments, the topically applied pharmaceutical composition further comprises pramoxine. In certain embodiments, the topically applied pharmaceutical composition comprises phenylephrine from about 0.05% (w/w) to about 1.0% (w/w). In some embodiments, the topically applied pharmaceutical composition further comprises glycerin. In other embodiments, the topically applied pharmaceutical composition further comprises white petrolatum. In certain embodiments, the topically applied pharmaceutical composition comprises glycerin from about 10% (w/w) to about 20% (w/w). In certain embodiments, the topically applied pharmaceutical composition comprises white petrolatum from about 10% (w/w) to about 20% (w/w). In certain embodiments, the topically applied pharmaceutical composition comprises pramoxine HCl from about 1.0% (w/w) to about 2.0% (w/w). In certain embodiments, the topically applied pharmaceutical composition further comprises light mineral oil or shark liver oil. In certain embodiments, the topically applied pharmaceutical composition comprises light mineral oil from about 10% (w/w) to about 20% (w/w). In certain embodiments, the topically applied pharmaceutical composition comprises shark liver oil from about 0.5% (w/w) to about 10% (w/w). In some embodiments, the muscular injury is selected from a muscle tear, muscle pull, muscle bruising, muscle swelling, and the like. In some embodiments, the muscular injury is selected from a muscle tear, muscle pull, muscle bruising, muscle strain or muscle swelling. In other embodiments, the phenylephrine is present as a pharmaceutically acceptable salt. In certain embodiments, the pharmaceutically acceptable salt is phenylephrine hydrochloride.

In some embodiments are provided methods of treating soft tissue injury in an individual comprising topical application of a pharmaceutical composition comprising phenylephrine. In some embodiments, the topically applied pharmaceutical composition further comprises glycerin. In other embodiments, the topically applied pharmaceutical composition further comprises white petrolatum. In other embodiments, the topically applied pharmaceutical composition further comprises pramoxine. In certain embodiments, the topically applied pharmaceutical composition comprises phenylephrine from about 0.05% (w/w) to about 1.0% (w/w). In certain embodiments, the topically applied pharmaceutical composition comprises glycerin from about 10% (w/w) to about 20% (w/w). In certain embodiments, the topically applied pharmaceutical composition comprises white petrolatum from about 10% (w/w) to about 20% (w/w). In certain embodiments, the topically applied pharmaceutical composition comprises pramoxine HCl from about 1.0% (w/w) to about 2.0% (w/w). In certain embodiments, the topically applied pharmaceutical composition further comprises light mineral oil or shark liver oil. In certain embodiments, the topically applied pharmaceutical composition comprises light mineral oil from about 10% (w/w) to about 20% (w/w). In certain embodiments, the topically applied pharmaceutical composition comprises shark liver oil from about 0.5% (w/w) to about 10% (w/w). In some embodiments, the muscular injury is selected from a muscle tear, muscle pull, muscle bruising, or muscle swelling. In other embodiments, the phenylephrine is present as a pharmaceutically acceptable salt. In certain embodiments, the pharmaceutically acceptable salt is phenylephrine hydrochloride.

In some embodiments, the vasoconstrictor is formulated into a variety of topically administrable compositions, such as solutions, suspensions, lotions, gels, pastes, medicated sticks, medicated bandages, rubs, scrubs, smears, balms, creams or ointments. Such pharmaceutical composition optionally contains solubilizers, stabilizers, tonicity enhancing agents, buffers and preservatives, as known in the art.

### EXAMPLES

### Formulation of Composition For Topical Administration

### Example 1

### Topical Gel Composition

To prepare a pharmaceutical topical gel composition, 100 mg of a vasoconstrictor such as phenylephrine is mixed with 1.75 g of hydroxypropyl celluose, 10 mL of propylene glycol, 10 mL of isopropyl myristate and 100 mL of purified alcohol USP. The resulting gel mixture is then incorporated into a container, such as a tube, which is suitable for topical administration.

### Example 2

### Topical Cream Composition

To prepare a pharmaceutical topical cream composition, 500 mg of phenylephrine is mixed with 1.75 g of hydroxypropyl celluose, 10 mL of propylene glycol, 10 mL of isopropyl myristate and 100 mL of water USP. The resulting mixture is place into a homogenizer and agitated until a cream-like emulsion is obtained. The resulting mixture is then incorporated into a container, such as a tube, which is suitable for dispensing a cream.

### Treament of Injury

### Example 3

### Treatment of Soft Tissue Injury Due To A Twisted Ankle

Symptoms: major pain, swelling, pooling of blood (hematoma) under the skin of the ankle and the top of the foot and the top of the toes. Symptoms disappeared after topical application of a cream comprising glycerin USP (14.4%), phenylephrine HCl USP (0.25%), pramoxine HCl USP (1%), and white petrolatum (15%) for 2-3 times per day over a period of two weeks. In comparison, other products were used for relief of these symptoms without significant, lasting or comparable success. Such products include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol.); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 4

### Treatment of Soft Tissue Injury Due To Severe Landing On The Foot: Bone Bruise of the Foot

Symptoms: major pain, pooling of blood (hematoma) under the skin. Symptoms disappeared after topical application of a cream comprising glycerin USP (14.4%), phenylephrine HCl USP (0.25%), pramoxine HCl USP (1%), and white petrolatum (15%) for 2-3 times per day over a period of 3 days. In comparison, other products previously used for relief of these symptoms without significant, lasting and comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol.); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 5

### Treatment of Sports-Related Injury Common Among Tennis Players: Tennis Elbow

Symptoms: pain and tenderness. Symptoms were alleviated after topical application of a cream comprising glycerin USP (14.4%), phenylephrine HCl USP (0.25%), pramoxine HCl USP (1%), and white petrolatum (15%) for 2 times per day for as long as needed. Other products previously used for relief of these symptoms without significant, lasting or comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol.); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 6

### Treatment of Sports-Related Injury Incurred During Fast Movement And Stop-And-Go During Tennis: Muscle Tear of Quadriceps

Symptoms: major pain, swelling, pooling of blood (hematoma) within the muscle. Symptoms disappeared after topical application of a cream comprising glycerin USP (14.4%), phenylephrine HCl USP (0.25%), pramoxine HCl USP (1%), and white petrolatum (15%) for 2-3 times per day over a period of 2 days. Other products previously used for relief of these symptoms without significant, lasting or comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol.); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 7

### Treatment of Sports-Related Injury Incurred During Fast Movement And Stop-And-Go During Tennis: Bone Bruise of the Knee

Symptoms: major pain, swelling, pooling of blood (hematoma) within the knee. Symptoms disappeared after topical application of a cream comprising glycerin USP (14.4%), phenylephrine HCl USP (0.25%), pramoxine HCl USP (1%), and white petrolatum (15%) for 2-3 times per day over a period of 2 days.

Other products previously used for relief of these symptoms without significant, lasting or comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 8

### Treatment of Sports-Related Injury: Shoulder Soreness

Symptoms: pain and tenderness. Symptoms were alleviated after topical application of a cream comprising glycerin USP (14.4%), phenylephrine HCl USP (0.25%), pramoxine HCl USP (1%), and white petrolatum (15%) for 2 times per day for as long as needed. Other products previously used for relief of these symptoms without significant, lasting or comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

### Example 9

### Treatment of Low Back Pain Due To Sports Injury, Muscle Overuse, Muscle Irritation, Muscle Strain, Muscle Ruptures, Muscle Tears, And Injury To Muscles And Ligaments

Symptoms were alleviated after topical application of a cream comprising glycerin USP (14.4%), phenylephrine HCl USP (0.25%), pramoxine HCl USP (1%), and white petrolatum (15%) for 2 times per day for as long as needed. Other products previously used for relief of these symptoms without significant, lasting or comparable success include “Traumeel Anti-inflammatory Analgesic” Homeopathic Ointment (Active ingredients: Each 100 g of ointment contains: Calendula officinalis 1×, Hamamelis virginiana 1×, Arnica montana, radix 3×1.50 g each; Aconitum napellus 3×, Belladonna 3×1.00 g each; Bellis perennis 1×, Chamomilla 1×, Echinacea angustifolia 1×, Echinacea purpurea 1×0.50 g each; Millefolium 1×0.30 g; Hepar sulphuris calcareum 8×0.25 g; Mercurius solubilis 8×0.12 g; Symphytum officinale 4×0.10 g; Hypericum perforatum 6×0.09 g. Inactive ingredients: Purified water, paraffin, white petrolatum, ethanol, and cetylstearyl alcohol); “This Works: Muscle Therapy” potent natural muscle lotion (active ingredients: essential oils including Marjoram, Black Pepper and Clove); Aleve “All Day Strong” caplets (Active ingredient: Naproxen sodium, 220 mg); Tylenol “Extra Strength” caplets (Active ingredient: Acetaminophen, 500 mg); Advil tablets (Active ingredient: Ibuprofen, 200 mg).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a soft tissue injury in an individual comprising topical application of a pharmaceutical composition comprising a vasoconstrictor and a topical analgesic.

2. The method of claim 1, wherein the soft tissue injury is a muscular injury, a sports-related injury or a muscle-related low back injury.

3. The method of claim 1, wherein the vasoconstrictor is phenylephrine and the topical analgesic is pramoxine.

4. The method of claim 2, wherein the topically applied pharmaceutical composition further comprises at least one of the following: menthol, methyl salicylate, or capsaicin.

5. The method of claim 2, wherein the topically applied pharmaceutical composition further comprises at least one of the following: aloe barbadebsis leaf extract, benzoic acid, butylated hydroxyanisole, carboxymethylcellulose sodium, cetyl alcohol, citric acid, dexpanthenol, edentate sodium, glyceryl stearate, laureth-23, methylparaben, mineral oil, panthenol, polyoxyl lauryl ether, polyoxyl stearyl ether, propyl gallate, propylene glycol, propylparaben, sodium benzoate, steareth-2, steareth-20, stearyl alcohol, tocopherol, vitamin E, xanthan gum, wintergreen, eucalyptus oil, corn oil, lanolin, mineral oil, thyme oil, or white wax.

6. A method of treating a soft tissue injury in an individual comprising topical application of a pharmaceutical composition comprising a phenylephrine.

7. The method of claim 6, wherein the soft tissue injury is a muscular injury, a sports-related injury or a muscle-related low back injury.

8. The method of claim 7, wherein muscular pain associates with said muscular injury.

9. The method of claim 6, wherein the topically applied pharmaceutical composition further comprises glycerin.

10. The method of claim 6, wherein the topically applied pharmaceutical composition further comprises white petrolatum.

11. The method of claim 6, wherein the topically applied pharmaceutical composition further comprises pramoxine.

12. The method of claim 6, wherein the topically applied pharmaceutical composition comprises phenylephrine from about 0.05% (w/w) to about 2.0% (w/w).

13. The method of claim 9, wherein the topically applied pharmaceutical composition comprises glycerin from about 10% (w/w) to about 20% (w/w).

14. The method of claim 10, wherein the topically applied pharmaceutical composition comprises white petrolatum from about 10% (w/w) to about 20% (w/w).

15. The method of claim 11, wherein the topically applied pharmaceutical composition comprises pramoxine HCl from about 1.0% (w/w) to about 2.0% (w/w).

16. The method of claim 6, wherein the topically applied pharmaceutical composition further comprises light mineral oil or shark liver oil.

17. The method of claim 16, wherein the topically applied pharmaceutical composition comprises light mineral oil from about 10% (w/w) to about 20% (w/w).

18. The method of claim 16, wherein the topically applied pharmaceutical composition comprises shark liver oil from about 0.5% (w/w) to about 10% (w/w).

19. The method of claim 7, wherein the muscle injury is selected from a muscle tear, muscle pull, muscle bruising, muscle strain or muscle swelling.

20. The method claim 19, wherein the injury occurs in a muscle selected from:
a. neck muscle comprising omohyoid, sternohyoid, sternocleidomastoid, or trapezius muscle;
b. chest muscle comprising pectoralis major, or pectoralis minor muscle;
c. shoulders muscles comprising deltoid, anterior head, middle head, or posterior head;
d. bicep muscle comprising brachialis, biceps brachil long head, or biceps brachil short head;
e. forearm muscle comprising extensor carpi ulnaris, palmaris longus, extensor pollicis brevis, abductore pollicis longus, extensor pollicis longus, flexor carpi radialis, brachioradialis, or flexor carpi ulnaris;
f. abdominal muscle comprising serratur anterior, external oblique, rectur abdominis, tendinous inscriptions, or external oblique;
g. thigh muscle comprising quadriceps, vastus lateralis, vastus rectus femoris, vastus medialis, vastus intermedialis; sartorius, pectineus, adductor longus, gracilis, or tensor fasciae latae;
h. calf muscle comprising gastrocnemius soleus, tibialis anterior, peroneus longus, extensor digitorum longus, peroneus brevis, or flexor hallucis longus;
i. tricep muscle comprising triceps brachi, triceps lateral head, triceps long head, or triceps medial head;
j. glute muscle comprising gluteus medius, or gluteus maximus; and
k. hamstring muscle comprising liotibial band, biceps femoris, adductor magnus, semitendinosus gracilis, or semimembranosus.

21. The method of claim 7, wherein the muscle-related low back injury occurs in at least one muscle selected from:
a. back muscles comprising teres minor, teres major, intraspinatus, rhomboid major, trapezius, erector spinea, latissimus dorsi, or thoracolumbar fascia;
b. lower back muscles comprising erector spinea, thoracolumbar fascia, external oblique, or gluteus medius; and
c. lower back muscles that are involved in low back pain caused by three types of muscles that support the spine, comprised of extensors (back muscles and gluteal muscles), flexors (abdominal muscles and iliopsoas muscles), obliques and rotators (side muscles).

22. The method of claim 6, wherein the injury is selected from:
a. hematomas associated with trauma-induced pooling of blood under the skin and within or around muscle tissue;
b. muscle bruising and muscle tears in skeletal muscles;
c. low back muscles;
d. bone bruises of the knee;
e. bone bruises of the shin;
f. bone bruises of the foot;
g. bone bruises of the ball of the foot;
h. bone bruises of the heel;
i. bone bruises of the toes;
j. bone bruises of the ankle;
k. sore elbow;
l. tennis elbow;
m. bone bruise of the elbow;
n. sore shoulder; and
o. bone bruise of the shoulder.
